(19) United States
(12) Patent Application Publication
(10) Pub. No.: US 2020/0237202 A9
(43) Pub. Date: Jul. 30, 2020

(54) DEVICE FOR VERIFYING PLACEMENT OF ENDOTRACHEAL, NASOGASTRIC, OROGASTRIC TUBES

(71) Applicant: ANGELE INNOVATIONS, LLC, San Marcos, CA (US)

(72) Inventor: Janice M. Shields, San Marcos, CA (US)

(21) Appl. No.: 15/684,666

(22) Filed: Aug. 23, 2017

(51) Int. Cl. A61B 1/07; A61B 1/06; A61B 1/00

(52) U.S. Cl. CPC A61B 1/07; A61B 1/0684; A61M 16/0465; A61B 2503/045; A61M 2240/00; A61B 1/00126

(57) ABSTRACT

A device for visually verifying the proper placement of a tube (e.g. a nasogastric tube) in the body of a neonate, essentially includes an optical fiber and a light source. In combination, the light source is connected to the proximal end of the optical fiber. The light source is then activated to illuminate the optical fiber, and the illuminated optical fiber is advanced through the tube into the body of the neonate. Due to the translucent nature of neonate body tissue, the optical fiber illuminates both the tube and anatomical features of the neonate's internal organs. With this optical capability, a visual determination can be made as to whether the tube has been properly placed.

### FIELD OF THE INVENTION

The present invention pertains generally to systems and methods that can be used to verify the proper placement of feeding and ventilating tubes in the body of a neonate. More particularly, the present invention pertains to systems and methods that implement optical techniques for visually verifying the proper placement of tubes in a neonate. The present invention is particularly, but not exclusively, useful as a system or method that advances an illuminated optical fiber through a prepositioned tube in the body of a neonate, to transmit light from the optical fiber in the tube and through the translucent body tissue of a neonate to provide visual verification of a proper placement of the tube in the body of the neonate.

### BACKGROUND OF THE INVENTION

It is well known and widely appreciated that neonates have unique medical needs that require special attention. Of continuing importance for all neonates, regardless of whatever else their needs might be, their basic needs for ventilation and nourishment persist and cannot be ignored. Moreover, it happens that these considerations can be of critical importance. For example, it is not uncommon for a neonate to be unable to either eat or breathe. In both instances some form of intervention is required, and most often this intervention requires the placement of a tube in the neonate.

Of crucial importance when tubes are used for a neonate is that they be properly placed. In particular, it is important that the tube be advanced through the esophagus and into the stomach, rather than through the larynx and into the lungs. Verifying proper tube placement, however, can be problematic for a variety of reasons. For the most part, the difficulties here arise from a basic inability to visually verify a proper tube placement. The problem is further exacerbated by the fact that due to the fragility of a neonate, x-ray techniques are preferably avoided.

With the above considerations in mind, the present invention has recognized that the body tissue of a neonate is relatively more translucent than that of more mature tissue. Indeed, it can be demonstrated that interior anatomical organs of a neonate can be visualized with light that has been passed through the body tissue of a neonate. Most importantly, this capability is sufficiently detailed to inspect tube placements in a neonate. With this in mind, the present invention recognizes that when a nasogastric tube is inadvertently inserted into the trachea of a neonate, rather than continuing on through the esophagus and into the stomach, the problem can be visually detected.

Accordingly, it is an object of the present invention to provide a system and method for verifying the placement of a tube in a neonate that implement optical techniques for visually verifying the proper placement of tubes in the neonate. Another object of the present invention is to provide a system or method that advances an illuminated optical fiber through a prepositioned tube in the body of a neonate, and to transmit light from the optical fiber while it is in the tube, through the translucent body tissue of a neonate, to thereby provide visual verification of a proper placement of the tube in the body of the neonate. Still another object of the present invention is to provide a system and method for verifying the placement of a tube in a neonate that is easy to use, is simple to manufacture, and is comparatively cost effective.

### SUMMARY OF THE INVENTION

In accordance with the present invention a device for verifying the placement of a tube in a neonate essentially includes an optical fiber and a light source. In combination, the light source is connected to the proximal end of the optical fiber. Preferably, this connection is provided by a swivel connector that will allow the light source to be selectively angled relative to the optical fiber, to thereby facilitate an unencumbered manipulation of the optical fiber during its use.

As intended for the present invention, the verification device is used to inspect and verify the placement of an intubation tube such as an endotracheal tube, a nasogastric tube or an orogastric tube (hereinafter each individually referred to simply as a “tube”). In each case the tube will have a length L_{tube} and it will be formed with a lumen having a diameter Dₗᵤₘₑₙ. For its interaction with the tube, the optical fiber of the device will have a length L_{fiber} and it will have an outer diameter D_{fiber}. In their comparison with each other, the length L_{tube} of the optical fiber is greater than the length L_{tube} of the tube, and the diameter of the optical fiber D_{fiber} is less than the lumen diameter Dₗᵤₘₑₙ of the tube (L_{fiber}>L_{tube} and D_{fiber}<Dₗᵤₘₑₙ).

It is an important aspect of the optical fiber for the device of the present invention that it be made of a material having a high light-loss characteristic. Stated differently, it is important for the optical fiber to radiate as much light as possible. In particular, it is important that the distal portion of the optical fiber have a high light radiating capability. To enhance this capability an alternate embodiment of the present invention envisions the possibility of enclosing the proximal portion of the optical fiber in an opaque sheath.

For purposes of the present invention, the lengthwise extent of the light radiating capability that is required for the distal portion of the optical fiber is determined by the length of the tube to be inspected. Preferably, the illuminated distal portion of the optical fiber will be longer than a predetermined intubation length. Specifically, for the present invention, an intubation length is equal to the length of the tube to be inspected, when the tube is positioned in the neonate for its particular purpose.

For an operation of the present invention, once a tube has been inserted into the neonate, the optical fiber of the device is illuminated by the light source. For this purpose, the light source is preferably a Light Emitting Diode (LED). The illuminated optical fiber is thereafter advanced into the tube. As the tube is being advanced, light that is radiated from the distal portion of the optical fiber passes from the optical fiber and through the tube. The light then continues to radiate from the tube through the body tissue of the neonate. As noted above, the passage of light through the body tissue of the neonate is due to the translucent nature of this tissue. The result is that the tube and the anatomical features of the neonate around the tube are both illuminated by the optical fiber. Thus, they can be visually observed together. Based on this observation a clinician will determine whether the tube has been properly placed. Once the inspection/verification of tube placement has been completed, the optical fiber is withdrawn from the tube, and the tube is repositioned, if necessary.

As an additional feature of the present invention, a cylindrical shaped supply container can be provided for holding a plurality of optical fibers. Structurally, the container will include a cylindrical shaped body portion that is formed with a plurality of tubular shaped cavities. Further, each cavity is aligned parallel to the axis of the cylindrical container, and each cavity is open at its top end and is closed at its bottom end in the container. Additionally, a cap is mounted for axial rotation on the top end of the container. For this combination, the cap is formed with a hole. Thus, as the cap is rotated, the hole is selectively position over a cavity in the body portion of the container for removing an optical fiber from the cavity.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of an optical device of the present invention;

FIG. 2 is a perspective view of a tube (e.g. a nasogastric tube) whose proper placement in a neonate can be inspected and verified by the device of the present invention;

FIG. 3 is a frontal view of a neonate, with a tube positioned in the nasopharynx and esophagus of the neonate, and with the optical device of the present invention inserted into the tube where it is illuminated to visually verify a proper placement of the tube in the neonate;

FIG. 4 is a cross-section view of the optical device inserted into the lumen of the tube as would be seen along the line 4-4 in FIG. 3; and

FIG. 5 is an exploded perspective view of a container for holding a plurality of optical devices for the present invention.

### DESCRIPTION OF THE PREFERRED EMBODIMENTS

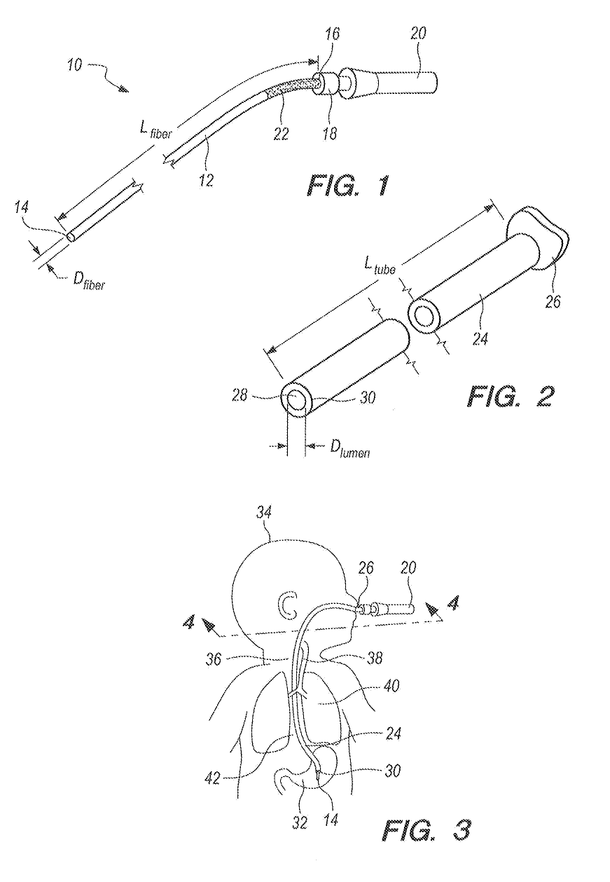

Referring initially to FIG. 1 a device in accordance with the present invention is shown and is generally designated 10. As shown, the device 10 includes an optical fiber 12 having a length L_{fiber} that extends between a distal end 14 and a proximal end 16 of the optical fiber 12. FIG. 1 also shows that a swivel connector 18 can be attached to the proximal end 16 of the optical fiber 12, and a light source 20 can be attached to the swivel connector 18. Functionally, the swivel connector 18 provides more flexibility for a user (not shown) when the device 10 is used for its intended purpose.

For the present invention, the light source 20 is preferably a source of light from a Light Emitting Diode of a type well known in the pertinent art. Further, the light source 20 can include a dimmer switch (not shown). It is also preferred that the optical fiber 12 be made of a high light-loss material, such as a clear plastic, so that the greatest amount of light will be radiated radially outwardly from the optical fiber 12 through a length of the optical fiber 12 with a predetermined distance from the distal end 14 of the optical fiber 12. Additionally, an opaque sheath 22 can be optionally provided on a portion of the optical fiber 12 that is proximal to this predetermined distance.

In accordance with the present invention, the predetermined distance along the optical fiber 12 where maximum light is to be radiated from the device 10 will be at least equal to the length L_{tube} of the tube 24 that is shown in FIG. 2. For purposes of the present invention, the tube 24 can be either an endotracheal tube, a nasogastric tube or an orogastric tube. In any case, as shown in FIG. 3, the tube 24 will have a length L_{tube} that is intubated during its use. Still referring to FIG. 2 it will be seen that the tube 24 includes an adapter 26 and it will be formed with a lumen 28 that extends along the entire length of the tube 24.

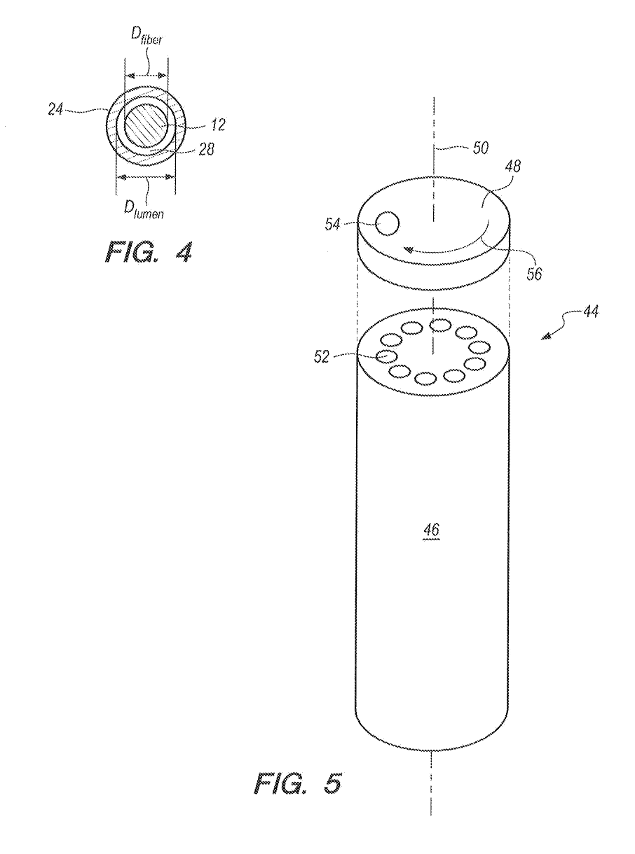

With cross reference between FIG. 1 and FIG. 2 it is to be appreciated that for the present invention, the optical fiber 12 will need to be inserted through the entire length L_{tube} of tube 24. In this combination, it will be necessary for the length L_{fiber} of optical fiber 12 to be greater than the length L_{tube} of tube 24. Further, as shown in FIG. 4, it will be necessary for the diameter D_{fiber} of optical fiber 12 to be less than the diameter Dₗᵤₘₑₙ of the tube 24 (L_{fiber}>L_{tube} and D_{fiber}<Dₗᵤₘₑₙ).

For a successful use of the device 10 when the tube 24 is either a nasogastric (NG) tube or an orogastric (OG) tube, FIG. 3 indicates it is imperative that the distal end 30 of the tube 24 be positioned in the stomach 32 of a neonate 34. Accordingly, this needs to be verified. For a proper placement of the tube 24, it is necessary that the tube 24 avoid the epiglottis 36 so it will bypass the larynx 38, and thereby prevent positioning the distal end 30 of tube 24 into the lungs 40. Instead, a proper placement of the tube 24 requires it be positioned through the esophagus 42 with its distal end 30 inserted into the stomach 32. The device 10 is intended to verify this proper placement. On the other hand, when tube 24 is an endotracheal tube (ETT), it is necessary to verify that the tube 24 has been appropriately directed into the lungs 40.

During a use of the device 10, the tube 24 is first advanced through the esophagus 42 until its distal end 30 is inserted into the stomach 32 of the neonate 34. The optical fiber 12 is then advanced through the lumen 28 of the tube 24. At this time, the light source 20 is activated to radiate light from the optical fiber 12. It then happens that, due to the translucent nature of tissue in the neonate 34, internal organs of the neonate 34 (e.g. the larynx 34, the esophagus 42, the lungs 40 and the stomach 32) are recognizable to the clinical personnel using the device 10. Of particular importance is the ability to visually determine whether the tube 24 has been properly advanced into the stomach 32 rather than into the lungs 40, or vice versa. After a proper placement of the tube 24 has been verified, the optical fiber 12 can be withdrawn.

As an accessory for the device 10, the present invention envisions the use of a supply container which is generally designated 44 in FIG. 5. As shown, the supply container 44 includes a cylindrical shaped body portion 46 and a cap 48 which, in combination, define a longitudinal axis 50. In this combination, the body portion 46 is formed with a plurality of cavities 52 which are each dimensioned and axially aligned to individually receive an optical fiber 12. Also, the cap 48 is formed with a single hole 54. In combination, the cap 48 is positioned over the cavities 52 in the body portion 46, and it is engaged with the body portion 46 for rotation about the axis 50 in the direction of arrow 56. Thus, as needed, the cap 48 can be selectively rotated to position the hole 54 of cap 48 over a cavity 52. An optical fiber 12 can then be retrieved from a cavity 52 of the supply container 44 for use in accordance with the purposes of the present invention.

While the particular Device for Verifying Placement of Endotracheal, Nasogastric, Orogastric Tubes as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for verifying the placement of a tube in a neonate, wherein the tube has a length L_{tube} and is formed with a lumen having a diameter Dₗᵤₘₑₙ, the device comprising:
an optical fiber having a proximal end and a distal end, wherein the optical fiber has a length, L_{fiber}, and an outer diameter, D_{fiber}, wherein L_{fiber} is greater than L_{tube} and D_{fiber} is less than Dₗᵤₘₑₙ (L_{fiber}>L_{tube} and D_{fiber}<Dₗᵤₘₑₙ); and
a light source, wherein the light source is optically attached to the proximal end of the optical fiber and is activated to transmit light through the optical fiber along the length L_{fiber} when the optical fiber is advanced into the lumen of the tube and through an intubation length of the tube, to position the distal end of the optical fiber in the neonate for visually verifying a proper placement of the tube in the neonate.

2. The device of claim 1 wherein the optical fiber is made of a high light-loss material to radiate light from the optical fiber along the length L_{fiber}.

3. The device of claim 1 wherein the optical fiber has a distal portion and a proximal portion and the distal portion has higher light-loss than the proximal portion.

4. The device of claim 3 wherein the proximal portion of the optical fiber is enclosed in an opaque sheath.

5. The device of claim 1 wherein the tube is selected from the group consisting of an endotracheal tube, a nasogastric tube and an orogastric tube.

6. The device of claim 1 wherein the light source is a Light Emitting Diode (LED).

7. The device of claim 1 further comprising a swivel connecter for optically attaching the light source to the optical fiber.

8. The device of claim 7 wherein the swivel connecter is mounted on the optical fiber at the proximal end thereof.

9. The device of claim 1 further comprising a supply container for holding a plurality of optical fibers, wherein the supply container comprises:
a cylindrical shaped body portion having a top end and a bottom end and defining a longitudinal axis therebetween, wherein the body portion is formed with a plurality of tubular shaped cavities with each cavity aligned parallel to the axis, and wherein each cavity is open at the top end of the body portion and closed at the bottom end of the body portion; and
a cap mounted for rotation on the top end of the body portion, wherein the cap is formed with a hole and the cap can be rotated on the body portion to selectively position the hole over a cavity in the body portion for removing an optical fiber from the cavity.

10. A system for treating a neonate which comprises:
a tube prepositioned in the neonate, wherein the tube has a length L_{tube} and is formed with a lumen having a diameter Dₗᵤₘₑₙ;
an optical fiber having a proximal end and a distal end, wherein the optical fiber has a length, L_{fiber}, and an outer diameter, D_{fiber}, wherein L_{fiber} is greater than L_{tube} and D_{fiber} is less than Dₗᵤₘₑₙ (L_{fiber}>L_{tube} and D_{fiber}<Dₗᵤₘₑₙ);
a light source, wherein the light source is optically attached to the proximal end of the optical fiber and is activated to transmit light through the optical fiber along the length L_{fiber}, and wherein the optical fiber is advanced into the lumen of the tube through an intubation length of the tube for visually verifying a proper placement of the tube in the neonate prior to treating the neonate; and
a supply container for holding a plurality of optical fibers for use in subsequent treatments of a neonate.

11. The system of claim 10 wherein the tube is selected from the group consisting of an endotracheal tube, a nasogastric tube and an orogastric tube.

12. The system of claim 10 wherein the optical fiber is made of a high light-loss material to radiate light from the optical fiber along the length L_{fiber}.

13. The system of claim 10 wherein the optical fiber has a distal portion and a proximal portion and the distal portion has higher light-loss than the proximal portion.

14. The system of claim 10 wherein the light source is a Light Emitting Diode (LED).

15. The system of claim 10 wherein the supply container further comprises:
a cylindrical shaped body portion having a top end and a bottom end and defining a longitudinal axis therebetween, wherein the body portion is formed with a plurality of tubular shaped cavities with each cavity aligned parallel to the axis, and wherein each cavity is open at the top end of the body portion and closed at the bottom end of the body portion; and
a cap mounted for rotation on the top end of the body portion, wherein the cap is formed with a hole and the cap can be rotated on the body portion to selectively position the hole over a cavity in the body portion for removing an optical fiber from the cavity.

16. A method for treating a neonate which comprises the steps of:
placing a tube in the neonate, wherein the tube has a length L_{tube} and is formed with a lumen having a diameter Dₗᵤₘₑₙ;
providing a device having an optical fiber and a light source, wherein the optical fiber has a proximal end and a distal end, with a length, L_{fiber}, and an outer diameter, D_{fiber}, wherein L_{fiber} is greater than L_{tube} and D_{fiber} is less then Dₗᵤₘₑₙ (L_{fiber}>L_{tube} and D_{fiber}<Dₗᵤₘₑₙ), and wherein the light source is optically attached to the proximal end of the optical fiber;
activating the light source to transmit light through the optical fiber along the length L_{fiber}; and
advancing the optical fiber into the lumen of the tube through an intubation length of the tube to position the distal end of the optical fiber in the neonate for visually verifying a proper placement of the tube in the neonate.

17. The method of claim 16 wherein the tube is selected from the group consisting of an endotracheal tube, a nasogastric tube and an orogastric tube.

18. The method of claim 16 wherein the optical fiber is made of a high light-loss material to radiate light from the optical fiber along the length L_{fiber}.

19. The method of claim 16 wherein the optical fiber has a distal portion and a proximal portion and the distal portion has higher light-loss than the proximal portion.

20. The system of claim 16 wherein the light source is a Light Emitting Diode (LED).
